(19) United States
(12) Patent Application Publication
(10) Pub. No.: US 2023/0094386 A1
(43) Pub. Date: Mar. 30, 2023

(54) Method and Device for Carrying out a qPCR Process

(71) Applicant: Robert Bosch GmbH, Stuttgart (DE)

(72) Inventors: Volker Fischer, Renningen (DE); Christoph Faigle, Muensing (DE); Torsten Long, Koeln (DE)

(21) Appl. No.: 17/904,301

(22) PCT Filed: Feb. 15, 2021
(86) PCT No.: PCT/EP2021/053656

§ 371 Date: Aug. 16, 2022

(30) Foreign Application Priority Data
Feb. 25, 2020 (DE) 10 2020 202 360.3

(51) Int. Cl. C12Q 1/6851; G16B 40/10; G16B 40/20

(52) U.S. Cl. CPC C12Q 1/6851; G16B 40/10; G16B 40/20

(57) ABSTRACT

The disclosure relates to a computer-implemented method for carrying out a quantitative polymerase chain reaction (qPCR) process, comprising the following steps: —cyclically carrying out qPCR cycles; —measuring an intensity value of a fluorescence relating to each qPCR cycle to obtain a qPCR curve from intensity values; —analyzing the shape of the qPCR curve using a data-based classification model trained to provide a classification result depending on the shape of the qPCR curve; and—carrying out the qPCR process depending on the classification result of the analysis of the shape of the qPCR curve.

### TECHNICAL FIELD

The invention relates to the use of a polymerase chain reaction method (PCR method), especially for detection of the presence of a pathogen. The present invention further relates to the evaluation of qPCR measurements.

### TECHNICAL BACKGROUND

DNA strand segments in a substance to be tested, such as, for example, a serum or the like, are detected by carrying out PCR methods in automated systems. Said PCR systems make it possible to amplify and detect particular DNA strand segments to be detected, for example those which can be assigned to a pathogen. A PCR method generally comprises cyclic use of the steps of denaturation, annealing and elongation. In particular, the PCR process involves splitting of a DNA double strand into individual strands and making each of them complete again by attachment of nucleotides in order to reproduce the DNA strand segments in each cycle.

The qPCR method makes it possible to quantify the pathogen load detected using this process. To this end, at least some of the nucleotides are provided with fluorescent molecules which, upon binding to the individual strand of the DNA strand segment to be detected, activate a fluorescence property. After synthesis of the double strands, a fluorescence value dependent on the number of DNA strand segments generated can be determined after each cycle.

During amplification, it is then possible to determine from the fluorescence values (intensity values) determined a qPCR curve which has a sigmoidal shape in the event of the presence of the DNA strand segment to be detected in the substance to be tested. In reality, the qPCR curves measured may contain artifacts, and so multiple parallel measurements are generally carried out in order to make a more accurate evaluation of the qPCR curves possible through averaging of the measurement values.

### DISCLOSURE OF THE INVENTION

According to the invention, a method for carrying out a qPCR method as claimed in claim 1 and a device and a qPCR system as claimed in the alternative independent claims are provided.

Further embodiments are specified in the dependent claims.

According to a first aspect, a method for conducting a quantitative polymerase chain reaction (qPCR) method is provided, comprising the following steps:
cyclic execution of qPCR cycles
measurement of an intensity value of a fluorescence after or during each qPCR cycle in order to obtain a qPCR curve composed of intensity values;
evaluation of the shape of the qPCR curve with the aid of a data-based classification model which has been trained to provide a classification result depending on the shape of the qPCR curve;
conduction of the qPCR method depending on the classification result from the evaluation of the shape of the qPCR curve.

The qPCR method comprises cyclic repetition of the steps of denaturation, annealing and elongation. In the case of denaturation, the entire double-stranded DNA in the substance to be tested is split into two individual strands at a high temperature. In the annealing step, one of the primers added to the substance is bound to the individual strands, which primers specify the starting point of amplification of the DNA strand segments to be detected. In the elongation step, a complementary second DNA strand segment is synthesized from free nucleotides on the individual strands provided with the primer. After each of these cycles, the DNA quantity of the DNA strand segments to be detected has thus ideally doubled.

By using the qPCR method, fluorescent molecules are incorporated as labels into the DNA strand segments to be detected, and so it is possible, via measurement of the intensity of the fluorescence after each elongation step, to determine a time plot of the intensity values. The qPCR curve thus obtained comprises three distinct phases, namely a baseline, in which the intensity of the fluorescence of the fluorescent light emitted by incorporated labels is still indistinguishable from the background fluorescence, an exponential phase, in which the fluorescence intensity rises above the baseline, i.e., becomes visible, the doubling of the DNA strands in each cycle causing the fluorescence signal to exponentially rise proportional to the quantity of the DNA strand segments to be detected, and a plateau phase, in which the reagents, i.e., the primer and the free nucleotides, are no longer present in the required concentration and no further doubling takes place.

For the detection of a specified DNA strand segment to be detected, which can correspond to a pathogen for example, the so-called ct (cycle threshold) value is relevant here. The ct value determines the start of the exponential phase and is determined by exceeding of a specific threshold, which has been defined for whichever DNA strand segment is to be detected and which is identical for all samples for the DNA strand segment to be detected, or is determined mathematically by the second derivative of the qPCR curve in the exponential phase and corresponds to the intensity value of the steepest rise of the qPCR curve. If the target value is known, the starting concentration of the DNA strand segment to be detected in the substance to be tested can be determined by back-calculation.

In reality, the qPCR curves are highly inaccurate and are subject to considerable fluctuations. Baseline drift can occur, which refers to the rise of the background fluorescence above the measurement cycles. This means that, even if no amplification is taking place, the fluorescence signal is rising. Further influencing factors which have an adverse effect on the accuracy of the qPCR curve can, for example, result from thermal noise, fluctuations or metering tolerances in the reagent concentration, and air pockets and artifacts in the fluorescence volume.

In conventional qPCR systems, what is done, firstly, is software-based correction of the PCR curves and what can be envisaged, secondly, is repeatedly measuring a sample under the same conditions and smoothing the resultant qPCR curves by averaging. However, this requires increased effort.

It is a concept of the above method to decide with the aid of a data-based classification model, especially in the form of an artificial neural network, especially a deep neural network or a recurrent neural network, especially an LSTM, or in the form of a support vector machine, whether a measured qPCR curve indicates that the DNA strand segment to be detected occurs or does not occur in the substance to be tested. Since measured qPCR curves are generally greatly affected by noise, use of the trained data-based classification model allows more reliable assessment of the qPCR curve shape, even in borderline cases, as to whether the DNA strand segment to be detected occurs or does not occur in the substance to be tested.

The classification model can have been trained to provide, depending on the qPCR curve, a classification result which indicates or does not indicate a presence of a DNA strand segment to be detected. In particular, it is possible to train the data-based classification model on labeled qPCR curves as training data, so that a measured qPCR curve can be appropriately classified. However, this requires extensive training of the classification model with a high number of data sets of manually classified or labeled qPCR curves, since no presuppositions are taken into account.

Furthermore, residual error plots between the measured qPCR curve and a parameterized presence function and a parameterized nonpresence function can be determined, wherein the classification model has been trained to provide, depending on at least one of the residual error plots, a classification result which indicates or does not indicate a presence of a DNA strand segment to be detected. Therefore, it is alternatively possible to fit the measured qPCR curve to parameterized ideal shapes of a qPCR curve in the case of a presence of a DNA strand segment to be detected (presence curve shape) in the substance to be tested and in the case of a nonpresence of a DNA strand segment to be detected (nonpresence curve shape) in the substance to be tested. This is done by parameterization of the presence curve shape and the nonpresence curve shape to the measured qPCR curve. The residual error plots between the parameterized presence curve shape and the parameterized nonpresence curve shape and the actually measured qPCR curve can then be evaluated with the aid of a trained data-based classification model. The classification model is trained to decide whether the respective curve fit is based on a parameterized ideal shape which comes closest to the measured shape of the qPCR curve. This means that, with the aid of the classification model, it is established whether the measured qPCR curve tends to correspond to a qPCR presence curve or qPCR nonpresence curve.

A presence of a DNA strand segment to be detected is established if the classification result based on the residual error plot from the parameterized presence function indicates the presence of the DNA strand segment to be detected. Accordingly, a nonpresence of the DNA strand segment to be detected can be established if the classification result based on the residual error plot from the parameterized nonpresence function indicates the nonpresence of the DNA strand segment to be detected.

The evaluation can be made by inferring a presence of the DNA strand segment to be detected if the classification result confirms a curve fit with the presence curve shape and does not confirm a curve fit with the nonpresence curve shape. Analogously, a nonpresence of the DNA strand segment to be detected is inferred if the classification result confirms a curve fit with the nonpresence curve shape and does not confirm a curve fit with the presence curve shape.

Whereas the first above-described variant avoids possible incorrect or inaccurate basic assumptions about the underlying typical curve shapes and can also depict unknown relationships and dynamics, the second above-described variant, owing to the available domain knowledge, can be used with a classification model which requires a less comprehensive training data set for its training.

Altogether, the use of a data-based classification model leads to a lower number of misclassifications compared to conventional methods.

It can be envisaged that the qPCR method is conducted by
signaling that a ct value is determinable and/or
determining the ct value from the parameterized presence function,

if a presence of the DNA strand segment to be detected is established.

According to a further aspect, a device for conducting a quantitative polymerase chain reaction (qPCR) method is, wherein the device is designed to execute the following steps:
cyclic execution of qPCR cycles
measurement of an intensity value of a fluorescence at each qPCR cycle in order to obtain a qPCR curve composed of intensity values;
evaluation of the shape of the qPCR curve with the aid of a data-based classification model which has been trained to provide a classification result depending on the shape of the qPCR curve;
conduction of the qPCR method depending on the classification result from the evaluation of the shape of the qPCR curve.

### BRIEF DESCRIPTION OF THE DRAWINGS

Embodiments will be more particularly elucidated below on the basis of the accompanying drawings, where:

FIG. 1 shows a schematic depiction of a cycle of a PCR method;

FIG. 2 shows a schematic depiction of a typical qPCR curve comprising a plot of intensity values;

FIG. 3 shows a measured plot of a qPCR curve;

FIGS. 4a and 4b show ideal plots of the qPCR curve in the case of a nondetectable substance and a detectable substance, respectively; and

FIG. 5 shows a flowchart to illustrate a method for conducting a qPCR measurement; and

FIG. 6 shows a flowchart to illustrate a further method for conducting a qPCR measurement.

### DESCRIPTION OF EMBODIMENTS

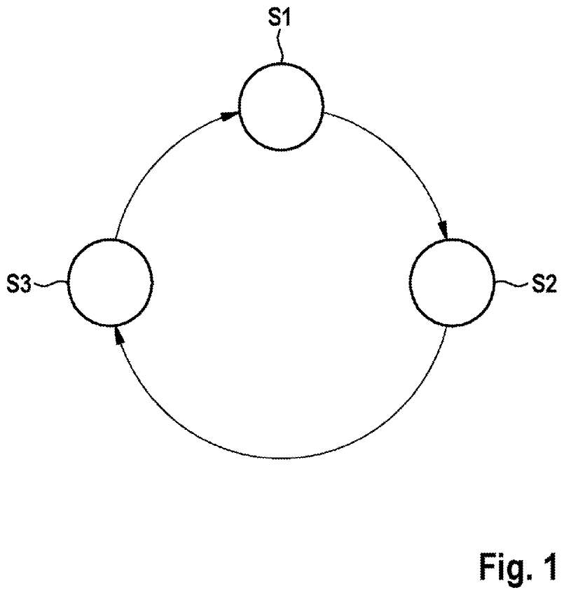

FIG. 1 shows a schematic depiction of a PCR method known per se, comprising the steps of denaturation, annealing and elongation.

In the annealing step S1, the double-stranded DNA in a substance is broken up into two individual strands at a high temperature of, for example, above 90° C. In a subsequent annealing step S2, a so-called primer is bound to the individual strands at a particular DNA position marking the start of a DNA strand segment to be detected. Said primer represents the starting point of an amplification of the DNA strand segment. In an elongation step S3, the complementary DNA strand segment is synthesized on the individual strands from free nucleotides added to the substance, starting at the position marked by the primer, with the result that the previously split individual strands have been completed to form complete double strands at the end of the elongation step.

By providing the free nucleotides or the primer with fluorescent molecules which exhibit fluorescence properties only when bound to the DNA strand segment, it is possible, by determining an intensity of a fluorescence following the elongation step S3, to obtain an intensity value of the fluorescence through an appropriate measurement. What is assigned to the measured intensity of the fluorescent light is an intensity value.

The method comprising steps S1 to S3 is executed cyclically and the intensity values are recorded in order to obtain a plot of intensity values as a qPCR curve.

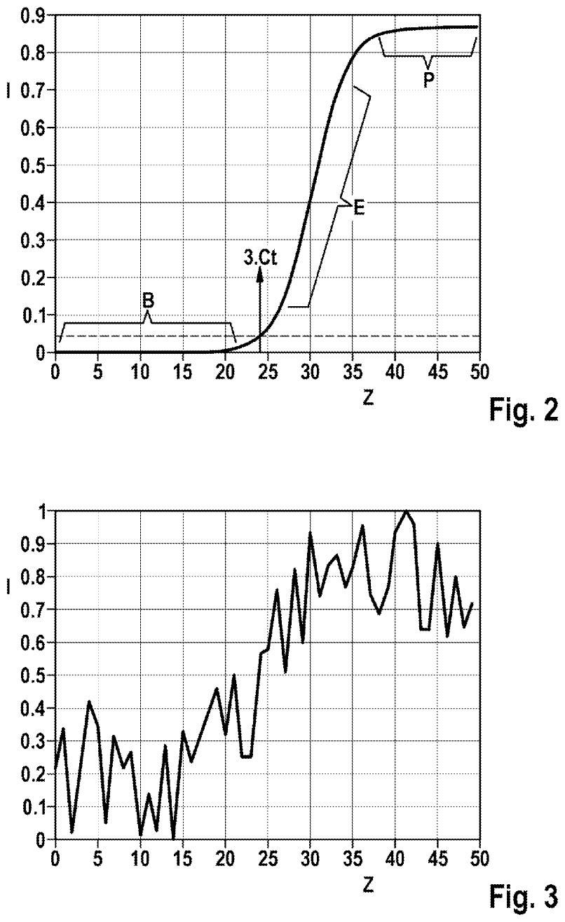

The plot of intensity values ideally has the shape depicted in FIG. 2. FIG. 2 shows a plot of normalized intensity I against the cycle index z. Said plot is divided into three sections, namely a baseline section B, in which the fluorescence of the incorporated fluorescent molecules is still indistinguishable from a background fluorescence, an exponential section E, in which the intensity values are visible and rise exponentially, and in a plateau section P, in which there is flattening of the rise in intensity values, since the reagents used (solution containing nucleotides) have been consumed and no further binding to broken-up individual strands is taking place.

FIG. 3 depicts, by way of example, a plot of the intensity values obtained in a real measurement as a qPCR curve. Strong fluctuations are evident, and these may result from background fluorescence, thermal noise, fluctuations in the reagent concentrations, and bubbles and artifacts in the fluorescence volume. It is evident that it is not readily possible to determine the baseline section, exponential section and plateau section of the qPCR curve.

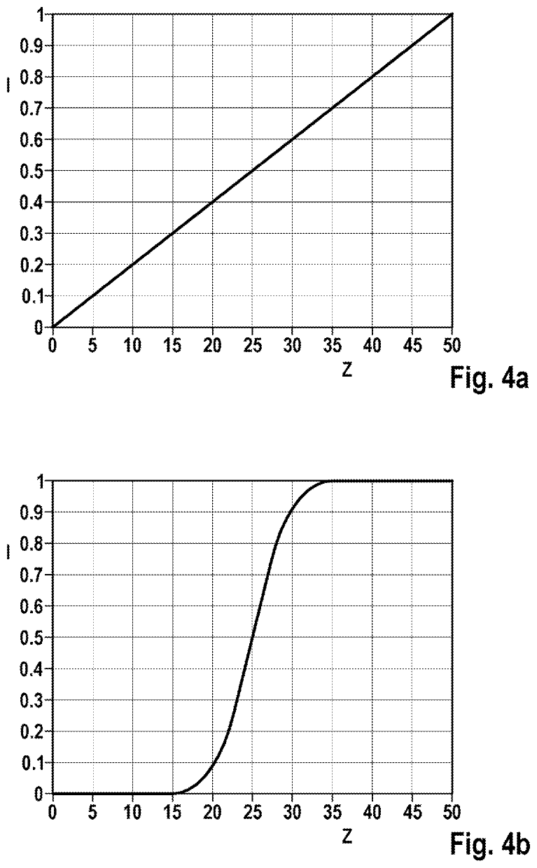

FIGS. 4a and 4b show ideal plots of a qPCR curve without the presence of a DNA strand segment to be detected and with the presence of a DNA strand segment to be detected, respectively.

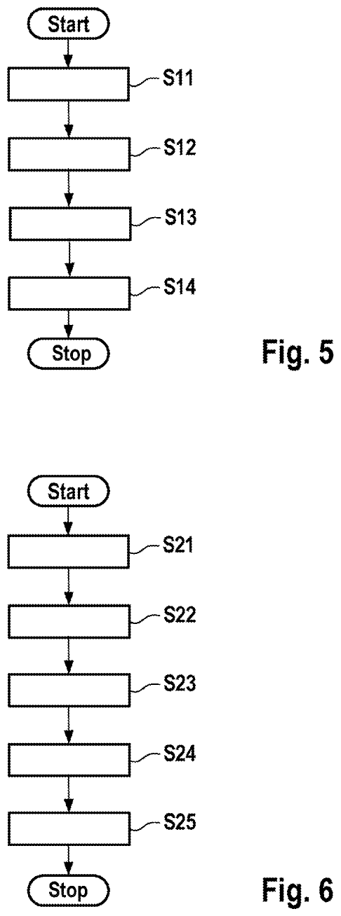

FIG. 5 depicts a flowchart to illustrate a method for conducting a qPCR measurement process. The method can be executed on a data processing device which controls a qPCR process on a PCR system and which provides from a PCR system in each cycle an intensity value indicating the intensity of a fluorescence depending on a DNA strand segment amplified during the qPCR. In the data processing device, the below-described method can be implemented in software and/or hardware.

In step S11, the qPCR measurement is carried out in order to receive intensity values in consecutive cycles of a qPCR measurement. The number of cycles for the qPCR measurement is about 30 to 60 cycles, preferably 40 cycles. A qPCR curve showing the intensity values (or values derived therefrom) against a cycle index is obtained.

In step S12, the intensity values of the qPCR curve are supplied to a trained classification model. The classification model is in the form of a data-based model, such as, for example, a SVM (support vector machine) or a deep neural network. Alternatively, the data-based classification model can also be formed with a neural network composed of temporal convolutional layers.

The classification model can have been trained with data sets of actually measured qPCR plots, each of which has been assigned a label indicating whether or not the data set (qPCR curve) corresponds to a measurement of a substance containing the DNA strand segment to be detected.

In step S13, it is determined, according to the result of the classification by the classification model, whether the qPCR curve corresponds to a presence or a nonpresence of the DNA strand segment to be detected, i.e., specified whether the DNA strand segment to be detected is present in the substance or not.

In step S14, the PCR method is executed according to the classification result. In particular, the qPCR method can be conducted by signaling that a ct value is determinable and determining the ct value from the parameterized presence function, if a presence of the DNA strand segment to be detected is established.

FIG. 6 depicts a flowchart to illustrate an alternative method for conducting the PCR measurement and for evaluating a measured qPCR curve.

In step S21, a qPCR method is used to carry out a qPCR measurement and to determine a qPCR curve through consecutive measurement of intensity values.

In step S22, a specified parametric nonpresence function is first parameterized by fitting the measured qPCR curve to the nonpresence function. For example, the nonpresence function can be a linear function, as depicted in FIG. 4a, which substantially corresponds to the plot characteristics of a baseline. Fitting to the nonpresence function can, for example, be done with the aid of least squares minimization.

In step S23, the measured qPCR curve is fitted to a presence function. The presence function corresponds to a parameterized function which substantially corresponds to plot characteristics as depicted in FIG. 4b. The presence function can have a sigmoid function. Parameterization of the presence function makes it possible to fit the measured qPCR curve to the presence function.

In step S24, residual error plots of the measured qPCR curve in relation to the parameterized presence function and in relation to the parameterized nonpresence function are determined.

In step S25, the residual error plots are supplied to a data-based classification model. The classification model has been trained on the basis of training data which assign residual error plots to the corresponding presence function or nonpresence function. This means that the training data indicate whether or not a residual error plot from the parameterized presence function confirms the presence of the strand segment to be detected. Furthermore, the training data indicate whether or not the residual error plot from the parameterized nonpresence function confirms the nonpresence of the strand segment to be detected.

Accordingly, it is established in step S25 with the aid of the residual error plot that a measured qPCR curve indicates a presence of the DNA strand segment to be detected in the substance if the classification model confirms the residual error plot from the presence function. Analogously, it is established with the aid of the residual error plot that a measured qPCR curve indicates a nonpresence of the DNA strand segment to be detected in the substance if the classification model confirms the residual error plot from the nonpresence function. This means that, if what generally arises from the residual error plot based on the parameterized presence function or the parameterized nonpresence function is that the classification result the residual error plot of the corresponding parameterized presence function or parameterized nonpresence function underlying the residual error plot, it is established that the DNA strand segment to be detected is present or is not present.

If the classification result with regard to the residual error plot gives rise to a result contrary to parameterized presence or nonpresence function underlying the residual error plot, it can be decided to discard the qPCR measurement, since it is not possible to make a clear decision about a presence or nonpresence of the DNA strand segment to be detected.

1. A method, which is computer implemented, for conducting a quantitative polymerase chain reaction (qPCR) process, the method comprising:
cyclically executing qPCR cycles;
measuring an intensity value of a fluorescence at each qPCR cycle to obtain a qPCR curve composed of intensity values;
evaluating a shape of the qPCR curve with a data-based classification model which has been trained to provide a classification result depending on the shape of the qPCR curve; and
conducting the qPCR process depending on the classification result from the evaluation of the shape of the qPCR curve.

2. The method as claimed in claim 1, wherein the data-based classification model comprises one of a neural network and a support vector machine model.

3. The method as claimed in claim 1, wherein the data-based classification model has been trained to provide, depending on the qPCR curve, the classification result which indicates one of a presence and a nonpresence of a DNA strand segment to be detected.

4. The method as claimed in claim 1 further comprising:
determining residual error plots between the qPCR curve and a parameterized presence function and a parameterized nonpresence function, the data-based classification model having been trained to provide, depending on at least one of the residual error plots, the classification result which indicates one of a presence and a nonpresence of a DNA strand segment to be detected.

5. The method as claimed in claim 4 further comprising:
establishing the presence of the DNA strand segment to be detected in response to the classification result based on the residual error plot from the parameterized presence function indicating the presence of the DNA strand segment to be detected.

6. The method as claimed in claim 4 further comprising:
establishing the nonpresence of the DNA strand segment to be detected in response to the classification result based on the residual error plot from the parameterized nonpresence function indicating the nonpresence of the DNA strand segment to be detected.

7. The method as claimed in claim 1, the conducting the qPCR process further comprising:
signaling that a ct value is determinable; and
determining the ct value from a parameterized presence function in response to a presence of the DNA strand segment to be detected being established.

8. A device for conducting a quantitative polymerase chain reaction process, the device being configured to:
cyclically execute qPCR cycles;
measure an intensity value of a fluorescence at each qPCR cycle to obtain a qPCR curve composed of intensity values;
evaluate a shape of the qPCR curve with a data-based classification model which has been trained to provide a classification result depending on the shape of the qPCR curve; and
conduct the qPCR process depending on the classification result from the evaluation of the shape of the qPCR curve.

9. The method as claimed in claim 1, wherein the method is carried out by executing a computer program.

10. A non-transitory electronic storage medium storing a computer program for conducting a quantitative polymerase chain reaction process, the computer program being configured to, when executed by a computer, cause the computer to:
cyclically execute qPCR cycles;
measure an intensity value of a fluorescence at each qPCR cycle to obtain a qPCR curve composed of intensity values;
evaluate a shape of the qPCR curve with a data-based classification model which has been trained to provide a classification result depending on the shape of the qPCR curve; and
conduct the qPCR process depending on the classification result from the evaluation of the shape of the qPCR curve.

11. The method as claimed in claim 2, wherein the data-based classification model comprises a deep neural network.

12. The method as claimed in claim 2, wherein the data-based classification model comprises a recurrent neural network.

13. The method as claimed in claim 12, wherein the recurrent neural network comprises an LSTM.
